(19) United States
(12) Patent Application Publication
(10) Pub. No.: US 2023/0093617 A1
(43) Pub. Date: Mar. 23, 2023

(54) CROSS-STRAP APPARATUS FOR OSTEOARTHRITIS BRACES

(71) Applicant: Townsend Industries, Inc.

(72) Inventor: Marie Camille DuPont

(73) Assignee: Townsend Industries, Inc., Bakersfield, CA (US)

(21) Appl. No.: 17/795,470

(22) PCT Filed: Feb. 3, 2021
(86) PCT No.: PCT/US2021/016489

§ 371 Date: Jul. 26, 2022

Related U.S. Application Data
(60) Provisional application No. 62/971,782, filed on Feb. 7, 2020.

(51) Int. Cl. A61F 5/01

(52) U.S. Cl. CPC A61F 5/0109

(57) ABSTRACT

A cross-strap apparatus is disclosed for use with a brace having an proximal portion engageable above a joint of a user, an opposing distal portion engageable below the joint, and first and second cross-straps extending between and interconnecting the proximal and distal portions, the first and second cross-straps transversely oriented relative to one another and positioned for intersecting one another in an overlapping fashion at a cross-strap intersection substantially at the joint. In at least one embodiment, the apparatus provides a resilient hub portion sized and configured for engagement with each of the first and second cross-straps proximal to the cross-strap intersection. The hub portion provides at least one first engagement mechanism positioned and configured for engagement with the first cross-strap, and at least one second engagement mechanism positioned and configured for engagement with the second cross-strap, thereby maintaining the position of the hub portion relative to the cross-strap intersection.

### RELATED APPLICATIONS

This application claims priority and is entitled to the filing date of U.S. Provisional Application Serial Number 62/971,782, filed on Feb. 7, 2020. The contents of the aforementioned application are incorporated herein by reference.

### BACKGROUND

The subject of this patent application relates generally to osteoarthritis braces, and more particularly to a cross-strap apparatus for an osteoarthritis brace configured for joining a pair of cross-straps of the brace, opposite a hinge of the brace, and allowing for independent movement of the cross-straps while maintaining a load directly across a center of the user’s joint during movement of the joint.

Applicant hereby incorporates herein by reference any and all patents and published patent applications cited or referred to in this application.

By way of background, braces are widely used to treat a variety of infirmities, including osteoarthritis. Such braces may be configured to impart forces or leverage on the limbs surrounding a joint in order to relieve compressive forces within a portion of the joint, or to reduce the load on the joint. Moreover, in the event that ligaments are weak and infirm, a brace may stabilize, protect, support, or rehabilitate the joint.

The knee is known to be one of the weakest joints in the body. Knee instability arising out of cartilage damage, ligament strain and other causes is relatively commonplace since the knee joint is subjected to significant loads during the course of almost any kind of physical activity requiring the use of the legs. One type of knee infirmity that many individuals are prone to having is unicompartmental osteoarthritis. Unicompartmental osteoarthritis may arise when there is a persistent uneven distribution of pressure in one of the medial and lateral compartments of the knee. Knee bracing is useful to provide compartment pain relief by reducing the load on the compartment through the application of an opposing external valgus or varus moment about the knee joint. Unloading knee braces have been shown to significantly reduce osteoarthritis knee pain while improving knee function.

There are many known unloading knee braces. Typically, braces of this type are designed to apply a moment about the knee through two mechanisms. The first mechanism is through the angulations of hinge components which induce a bending moment at a hinge. The second mechanism is provided by a three-point bending system via a pair of force straps that spirals around the knee while crossing over one another, applying a force to a prescribed aspect of the knee. However, it has been found that if more unloading of the knee is required by the brace than is obtained from normal strap tension, and if the force straps are further tightened, the knee is drawn towards the hinge and might strike the hinge. This results in the hinge applying forces to the knee that counteract the force applied by the force straps. In turn, the additional tightening of the force straps is mitigated or negated by the force exerted onto the knee from the hinge. Additionally, while some amount of movement from the force straps can be beneficial, it has been found that the force straps have a tendency to unintentionally move too far out of position, slipping above, below or to the side of the knee. Thus, there remains a need for an apparatus capable of allowing the independent movement of the force straps while maintaining a load directly across the center of the knee joint during gait.

Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a cross-strap apparatus for use with a brace, the brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint. In at least one embodiment, the apparatus provides a resilient hub portion sized and configured for engagement with each of the first and second cross-straps proximal to the cross-strap intersection. The hub portion provides an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection. During use, the apparatus is capable of allowing for independent movement of the first and second cross-straps while maintaining a load directly across a center of the joint during movement of the joint.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a perspective view of an exemplary cross-strap apparatus engaged with an exemplary osteoarthritis brace, in accordance with at least one embodiment;

FIG. 2 is a partial perspective view of the exemplary cross-strap apparatus, in accordance with at least one embodiment;

FIG. 3 is a further partial perspective view thereof, illustrating the engagement therewith of a pair of exemplary cross-straps, in accordance with at least one embodiment;

FIG. 4 is a further partial perspective view thereof, in accordance with at least one embodiment;

FIG. 5 is a partial perspective view of a further exemplary cross-strap apparatus, illustrating the engagement therewith of a pair of exemplary cross-straps, in accordance with at least one embodiment;

FIG. 6 is a partial perspective view of a still further exemplary cross-strap apparatus, illustrating the engagement therewith of a pair of exemplary cross-straps, in accordance with at least one embodiment; and

FIG. 7 is a partial perspective view of a still further exemplary cross-strap apparatus, illustrating the engagement therewith of a pair of exemplary cross-straps, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

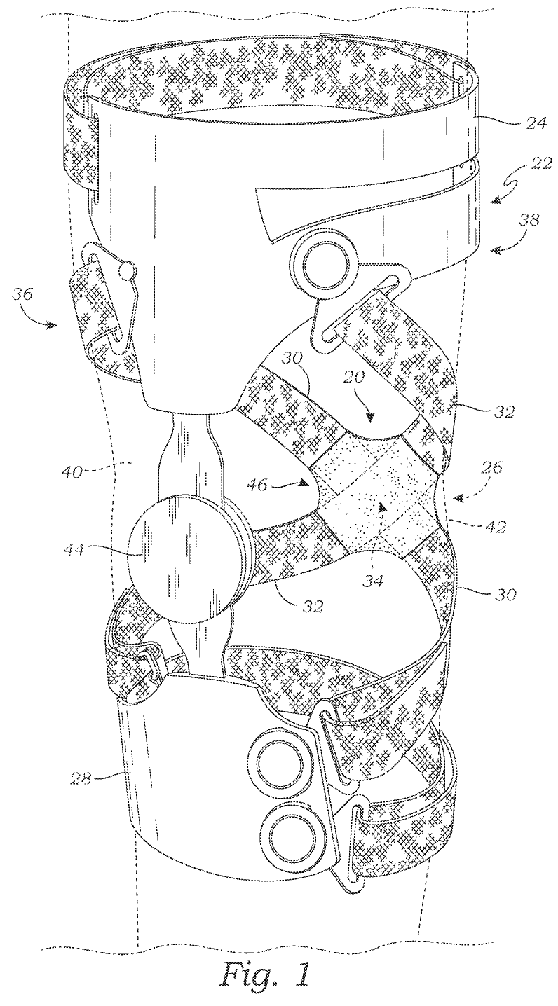

Turning now to FIG. 1, there is shown a perspective view of an exemplary embodiment of a cross-strap apparatus 20 engaged with an exemplary osteoarthritis brace 22. At the outset, it should be noted that while the apparatus 20 is discussed herein in the context of a knee brace in at least one embodiment, in further embodiments, the apparatus 20 may be utilized in combination with any other type of osteoarthritis brace (i.e., knee brace, elbow brace, etc.) now known or later developed, so long as said braces have at least one pair of cross-straps with which the apparatus 20 may be engaged. Thus, use of the present invention should not be read as being limited to only knee braces, nor even the specific brace 22 depicted in the drawings for illustrative purposes.

With continued reference to FIG. 1, in at least one embodiment, the exemplary brace 22 itself provides an proximal portion 24 configured for being engaged above a joint 26 of the user (such as a knee, for example), and an opposing distal portion 28 configured for being engaged below the user’s joint 26. Extending between and interconnecting the proximal and distal portions 24 and 28 is an at least one first cross-strap 30 and an at least one second cross-strap 32 transversely oriented relative to the first cross-strap 30 and positioned for intersecting the first cross-strap 30 in an overlapping fashion at a cross-strap intersection 34 substantially at the user’s joint 26. In at least one embodiment, the first and second cross-straps 30 and 32 are positioned on either a lateral side 36 or a medial side 38 of the brace 22, such that the cross-strap intersection 34 is substantially in contact with a lateral side 40 or medial side 42 of the user’s joint 26. In at least one such embodiment, the brace 22 may further provide a hinge 44 extending between the proximal and distal portions 24 and 28 and positioned on the other of the lateral or medial side 40 or 42 of the user’s joint 26, opposite the cross-strap intersection 34.

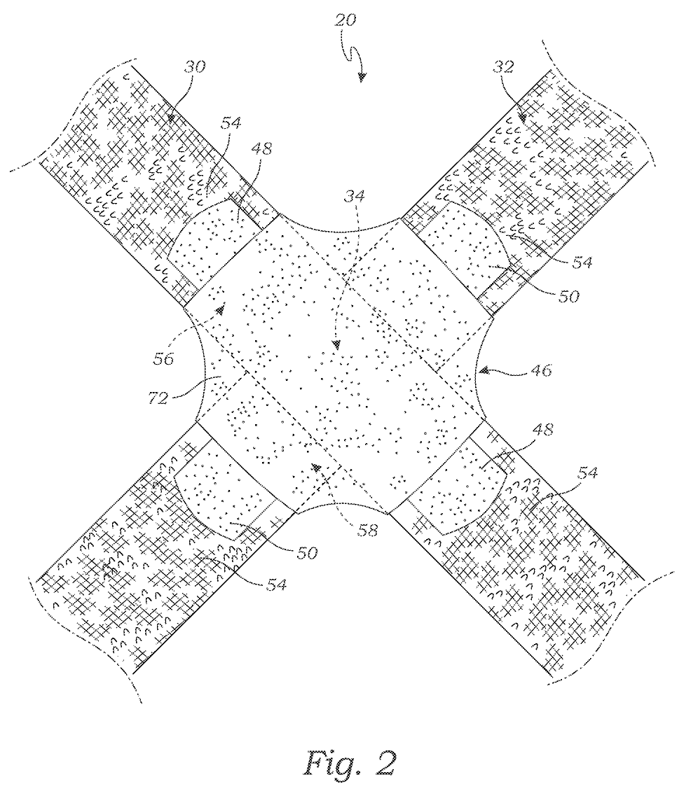

In at least one embodiment, as illustrated in FIG. 2, the apparatus 20 provides a resilient hub portion 46 sized and configured for engagement with each of the first and second cross-straps 30 and 32 proximal to the cross-strap intersection 34, such that the hub portion 46 is positioned in adjacent contact with the cross-strap intersection 34, thereby resiliently maintaining the position of the cross-strap intersection 34 relative to the first and second cross-straps 30 and 32 which allows for independent movement of the first and second cross-straps 30 and 32 as the user moves the joint 26, while always biasing the first and second cross-straps 30 and 32 back into their original positions. In other words, in at least one embodiment, the apparatus 20 acts as a locator to find a center of rotation of the joint 26 for ease of use. In at least one embodiment, the hub portion 46 is constructed out of one or more of fabric, silicone, rubber, soft plastic, or any other material (or combination of materials) – now known or later developed – having sufficient elastic properties for allowing the hub portion 46 to substantially carry out the functionality described herein. It should be noted that the specific sizes, shapes and dimensions of the hub portion 46 as depicted in the drawings are merely exemplary and are shown for illustrative purposes. In further embodiments, the hub portion 46 may take on any other sizes, shapes and/or dimensions – now known or later developed – capable of allowing the apparatus 20 to substantially carry out the functionality described herein.

In at least one embodiment, the hub portion 46 provides an at least one first engagement mechanism 48 positioned and configured for engagement with the first cross-strap 30, and an at least one second engagement mechanism 50 positioned and configured for engagement with the second cross-strap 32, thereby maintaining the position of the hub portion 46 relative to the cross-strap intersection 34 after the desired position of the hub portion 46 has been set, so that the cross-strap intersection 34 is thereafter unable to substantially move relative to the hub portion 46. In at least one embodiment, as illustrated in FIG. 2, the hub portion 46 provides a pair of first engagement mechanisms 48 positioned on opposing edges of the hub portion 46, along with a pair of second engagement mechanisms 50 positioned on further opposing edges of the hub portion 46 so as to be transversely oriented relative to the pair of first engagement mechanisms 48. In at least one embodiment, as illustrated best in FIG. 4, each of the first and second engagement mechanisms 48 and 50 provides hook material 52, while the first and second cross-straps 30 and 32 provide corresponding loop material 54. In at least one alternate embodiment, each of the first and second engagement mechanisms 48 and 50 provides loop material, while the first and second cross-straps 30 and 32 provide corresponding hook material. In still further embodiments, any other non-permanent engagement mechanism (or combination of mechanisms) – now known or later developed –may be substituted, such as snaps, buttons, buckles, magnets, zippers, etc. In such embodiments, the removable engagement of the first and second engagement mechanisms 48 and 50 allows for selective adjustment of the first and second engagement mechanisms 48 and 50 relative to the first and second cross-straps 30 and 32, respectively – which, in turn, allows for selective adjustment of the cross-strap intersection 34 relative to the hub portion 46. In still further embodiments, the first and second engagement mechanisms 48 and 50 are permanently engaged with the respective first and second cross-straps 30 and 32 using any permanent engagement mechanism (or combination of mechanisms) – now known or later developed –such as stitching, welding, adhesives, etc. It should be noted that the specific sizes, shapes, dimensions, types, quantities and relative positions of the first and second engagement mechanisms 48 and 50 as depicted in the drawings are merely exemplary and are shown for illustrative purposes. In further embodiments, the first and second engagement mechanisms 48 and 50 may take on any other sizes, shapes, dimensions, types, quantities and/or relative positions – now known or later developed – capable of allowing the apparatus 20 to substantially carry out the functionality described herein. In at least one such further embodiment, the first and second engagement mechanisms 48 and 50 are one and the same –i.e., a single engagement mechanism positioned and configured for engagement with each of the first and second cross straps 30 and 32, thereby maintaining the position of the hub portion 46 relative to the cross-strap intersection 34 after the desired position of the hub portion 46 has been set, so that the cross-strap intersection 34 is thereafter unable to substantially move relative to the hub portion 46. Additionally, in at least one alternate embodiment, the hub portion 46 is constructed out of a relatively non-resilient material, while each of the first and second engagement mechanisms 48 and 50 are constructed out of a relatively resilient material, thereby resiliently maintaining the position of the cross-strap intersection 34 relative to the first and second cross-straps 30 and 32.

In at least one embodiment, the hub portion 46 provides a first channel 56 sized and configured for slidably receiving the first cross-strap 30 therethrough, along with a second channel 58 sized and configured for slidably receiving the second cross-strap 32 therethrough so as to be transversely oriented relative to the first cross-strap 30. In at least one such embodiment, the first channel 56 provides an entry opening 60 sized and positioned for allowing a terminal end 62 of the first cross-strap 30 to pass therethrough as the first cross-strap 30 is inserted into the first channel 56, along with an opposing exit opening 64 sized and positioned for allowing the terminal end 62 of the first cross-strap 30 to pass therethrough as the terminal end 62 of the first cross-strap 30 subsequently exits the first channel 56. Similarly, in at least one such embodiment, the second channel 58 provides an entry opening 66 sized and positioned for allowing a terminal end 68 of the second cross-strap 32 to pass therethrough as the second cross-strap 32 is inserted into the second channel 58, along with an opposing exit opening 70 sized and positioned for allowing the terminal end 68 of the second cross-strap 32 to pass therethrough as the terminal end 68 of the second cross-strap 32 subsequently exits the second channel 58.

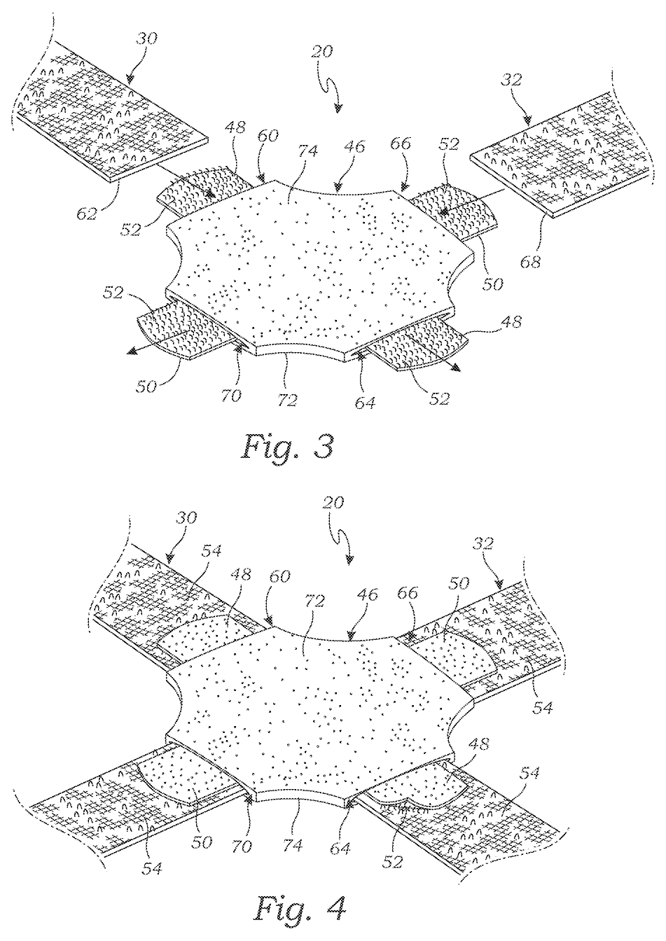

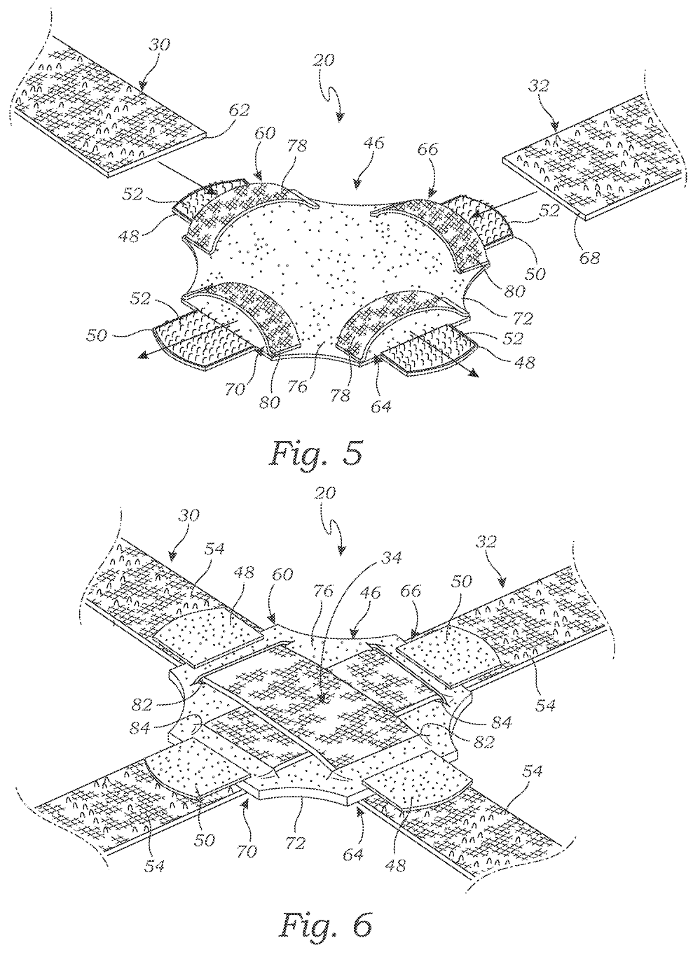

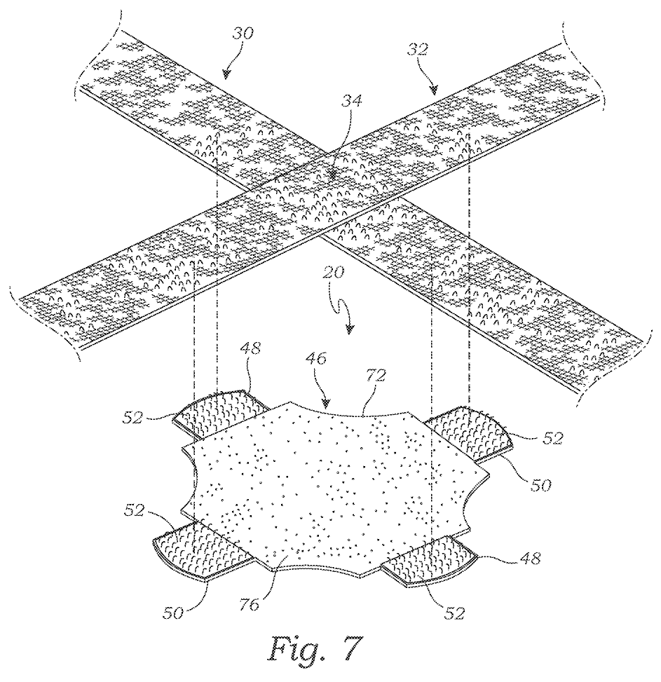

In at least one embodiment, as illustrated best in FIGS. 2 and 3, the hub portion 46 comprises a first layer 72 and an opposing second layer 74, with the first and second layers 72 and 74 cooperating to define the first and second channels 56 and 58 in a space therebetween, such that the cross-strap intersection 34 is positioned between the first and second layers 72 and 74 within the hub portion 46. In at least one such embodiment, the space between the first and second layers 72 and 74 is a unitary space, such that the first and second channels 56 and 58 are substantially coterminous therewithin. In at least one alternate embodiment, as illustrated in FIG. 5, the second layer 74 of the hub portion 46 is omitted, and a surface 76 of the first layer 72 provides a pair of spaced apart first loops 78 positioned and configured for defining the first channel 56, along with a pair of spaced apart second loops 80 positioned and configured for defining the second channel 58. In at least one further alternate embodiment, as illustrated in FIG. 6, the second layer 74 of the hub portion 46 is omitted, and the surface 76 of the first layer 72 provides a pair of spaced apart first slots 82 positioned and configured for defining the first channel 56, along with a pair of spaced apart second slots 84 positioned and configured for defining the second channel 58. It should be noted that the specific sizes, shapes, dimensions, structures and relative positions of the first and second channels 56 and 58 as depicted in the drawings are merely exemplary and are shown for illustrative purposes. In further embodiments, the first and second channels 56 and 58 may take on any other sizes, shapes, dimensions, structures and/or relative positions – now known or later developed – capable of allowing the apparatus 20 to substantially carry out the functionality described herein. Similarly, the specific sizes, shapes, dimensions, structures, quantities and relative positions of the first and second loops 78 and 80 (and, similarly, the first and second slots 82 and 84) as depicted in the drawings are merely exemplary and are shown for illustrative purposes. In further embodiments, the first and second loops 78 and 80 (and, similarly, the first and second slots 82 and 84) may take on any other sizes, shapes, dimensions, structures, quantities and/or relative positions –now known or later developed – capable of allowing the apparatus 20 to substantially carry out the functionality described herein. In at least one still further alternate embodiment, as illustrated in FIG. 7, the first and second channels 56 and 58 are omitted altogether, such that the first and second cross-straps 30 and 32 are simply engaged with the respective first and second engagement mechanisms 48 and 50.

In at least one embodiment, where the hub portion 46 provides first and second channels 56 and 58, the at least one first engagement mechanism 48 is positioned proximal at least one of the entry opening 60 and exit opening 64 of the first channel 56. In at least one such embodiment, one of first engagement mechanisms 48 is positioned proximal the entry opening 60 of the first channel 56, while another one of the first engagement mechanisms 48 is positioned proximal the exit opening 64 of the first channel 56. Similarly, in at least one embodiment, the at least one second engagement mechanism 50 is positioned proximal at least one of the entry opening 66 and exit opening 70 of the second channel 58. In at least one such embodiment, one of second engagement mechanisms 50 is positioned proximal the entry opening 66 of the second channel 58, while another one of the second engagement mechanisms 50 is positioned proximal the exit opening 70 of the second channel 58.

Accordingly, in at least one embodiment, the apparatus 20 is capable of allowing for independent movement of the first and second cross-straps 30 and 32 while maintaining a load directly across a center of the user’s joint 26 during movement of the joint 26. Thus, in at least one embodiment, where the brace 22 is a knee brace, the apparatus 20 is capable of offloading the knee joint 26 to relieve unicompartmental osteoarthritis; though it may also be used for other purposes, such as for preventing elbow or knee hyperextension, for example.

Aspects of the present specification may also be described as the following embodiments:
1. A cross-strap apparatus for use with a brace, the brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint, the apparatus comprising: a resilient hub portion sized and configured for engagement with each of the first and second cross-straps proximal to the cross-strap intersection; and the hub portion providing an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection; whereby, during use, the apparatus is capable of allowing for independent movement of the first and second cross-straps while maintaining a load directly across a center of the joint during movement of the joint.
2. The cross-strap apparatus according to embodiment 1, wherein hub portion is constructed out of one or more of fabric, silicone, rubber, and soft plastic.
3. The cross-strap apparatus according to embodiments 1-2, wherein: the hub portion provides a pair of first engagement mechanisms positioned on opposing edges of the hub portion; and the hub portion further provides a pair of second engagement mechanisms positioned on further opposing edges of the hub portion so as to be transversely oriented relative to the pair of first engagement mechanisms.
4. The cross-strap apparatus according to embodiments 1-3, wherein: the at least one first engagement mechanism is removably engageable with the first cross-strap; and the at least one second engagement mechanism is removably engageable with the second cross-strap.
5. The cross-strap apparatus according to embodiments 1-4, wherein each of the first and second engagement mechanisms provides hook material configured for engagement with corresponding loop material provided by each of the first and second cross-straps.
6. The cross-strap apparatus according to embodiments 1-5, wherein each of the first and second engagement mechanisms provides loop material configured for engagement with corresponding hook material provided by each of the first and second cross-straps.
7. The cross-strap apparatus according to embodiments 1-6, wherein the first and second engagement mechanisms are selected from at least one of snaps, buttons, buckles, magnets and zippers.
8. The cross-strap apparatus according to embodiments 1-7, wherein: the at least one first engagement mechanism is permanently engaged with the first cross-strap; and the at least one second engagement mechanism is permanently engaged with the second cross-strap.
9. The cross-strap apparatus according to embodiments 1-8, wherein: the hub portion provides a first channel sized and configured for slidably receiving the first cross-strap therethrough; and the hub portion further provides a second channel sized and configured for slidably receiving the second cross-strap therethrough so as to be transversely oriented relative to the first cross-strap.
10. The cross-strap apparatus according to embodiments 1-9, wherein: the first channel provides an entry opening sized and positioned for allowing a terminal end of the first cross-strap to pass therethrough as the first cross-strap is inserted into the first channel, and an opposing exit opening sized and positioned for allowing the terminal end of the first cross-strap to pass therethrough as the terminal end of the first cross-strap subsequently exits the first channel; and the second channel provides an entry opening sized and positioned for allowing a terminal end of the second cross-strap to pass therethrough as the second cross-strap is inserted into the second channel, and an opposing exit opening sized and positioned for allowing the terminal end of the second cross-strap to pass therethrough as the terminal end of the second cross-strap subsequently exits the second channel.
11. The cross-strap apparatus according to embodiments 1-10, wherein: the hub portion comprises a first layer and an opposing second layer, the first and second layers cooperating to define the first and second channels therebetween; whereby, during use of the apparatus, the cross-strap intersection is positioned between the first and second layers within the hub portion.
12. The cross-strap apparatus according to embodiments 1-11, wherein: the hub portion provides a pair of spaced apart first loops positioned and configured for defining the first channel; and the hub portion further provides a pair of spaced apart second loops positioned and configured for defining the second channel.
13. The cross-strap apparatus according to embodiments 1-12, wherein: the hub portion provides a pair of spaced apart first slots positioned and configured for defining the first channel; and the hub portion further provides a pair of spaced apart second slots positioned and configured for defining the second channel.
14. The cross-strap apparatus according to embodiments 1-13, wherein: the at least one first engagement mechanism is positioned proximal at least one of the entry opening and exit opening of the first channel; and the at least one second engagement mechanism is positioned proximal at least one of the entry opening and exit opening of the second channel.
15. The cross-strap apparatus according to embodiments 1-14, wherein: a first of the at least one first engagement mechanism is positioned proximal the entry opening of the first channel; a second of the at least one first engagement mechanism is positioned proximal the exit opening of the first channel; a first of the at least one second engagement mechanism is positioned proximal the entry opening of the second channel; and a second of the at least one second engagement mechanism is positioned proximal the exit opening of the second channel.
16. A cross-strap apparatus for use with a brace, the brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint, the apparatus comprising: a resilient hub portion sized and configured for engagement with each of the first and second cross-straps proximal to the cross-strap intersection; the hub portion providing an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection; the hub portion providing a first channel sized and configured for slidably receiving the first cross-strap therethrough; and the hub portion providing a second channel sized and configured for slidably receiving the second cross-strap therethrough so as to be transversely oriented relative to the first cross-strap in an overlapping fashion; whereby, during use, the apparatus is capable of allowing for independent movement of the first and second cross-straps while maintaining a load directly across a center of the joint during movement of the joint.
17. A combination cross-strap apparatus and brace comprising: a brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint; and a cross-strap apparatus comprising: a resilient hub portion sized and configured for engagement with each of the first and second cross-straps proximal to the cross-strap intersection; and the hub portion providing an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection; whereby, during use, the apparatus is capable of allowing for independent movement of the first and second cross-straps while maintaining a load directly across a center of the joint during movement of the joint.
18. The combination cross-strap apparatus and brace according to embodiment 17, wherein hub portion is constructed out of one or more of fabric, silicone, rubber, and soft plastic.
19. The combination cross-strap apparatus and brace according to embodiments 17-18, wherein: the hub portion provides a pair of first engagement mechanisms positioned on opposing edges of the hub portion; and the hub portion further provides a pair of second engagement mechanisms positioned on further opposing edges of the hub portion so as to be transversely oriented relative to the pair of first engagement mechanisms.
20. The combination cross-strap apparatus and brace according to embodiments 17-19, wherein: the at least one first engagement mechanism is removably engageable with the first cross-strap; and the at least one second engagement mechanism is removably engageable with the second cross-strap.
21. The combination cross-strap apparatus and brace according to embodiments 17-20, wherein each of the first and second engagement mechanisms provides hook material configured for engagement with corresponding loop material provided by each of the first and second cross-straps.
22. The combination cross-strap apparatus and brace according to embodiments 17-21, wherein each of the first and second engagement mechanisms provides loop material configured for engagement with corresponding hook material provided by each of the first and second cross-straps.
23. The combination cross-strap apparatus and brace according to embodiments 17-22, wherein the first and second engagement mechanisms are selected from at least one of snaps, buttons, buckles, magnets and zippers.
24. The combination cross-strap apparatus and brace according to embodiments 17-23, wherein: the at least one first engagement mechanism is permanently engaged with the first cross-strap; and the at least one second engagement mechanism is permanently engaged with the second cross-strap.
25. The combination cross-strap apparatus and brace according to embodiments 17-24, wherein: the hub portion provides a first channel sized and configured for slidably receiving the first cross-strap therethrough; and the hub portion further provides a second channel sized and configured for slidably receiving the second cross-strap therethrough so as to be transversely oriented relative to the first cross-strap.
26. The combination cross-strap apparatus and brace according to embodiments 17-25, wherein: the first channel provides an entry opening sized and positioned for allowing a terminal end of the first cross-strap to pass therethrough as the first cross-strap is inserted into the first channel, and an opposing exit opening sized and positioned for allowing the terminal end of the first cross-strap to pass therethrough as the terminal end of the first cross-strap subsequently exits the first channel; and the second channel provides an entry opening sized and positioned for allowing a terminal end of the second cross-strap to pass therethrough as the second cross-strap is inserted into the second channel, and an opposing exit opening sized and positioned for allowing the terminal end of the second cross-strap to pass therethrough as the terminal end of the second cross-strap subsequently exits the second channel.
27. The combination cross-strap apparatus and brace according to embodiments 17-26, wherein: the hub portion comprises a first layer and an opposing second layer, the first and second layers cooperating to define the first and second channels therebetween; whereby, during use of the apparatus, the cross-strap intersection is positioned between the first and second layers within the hub portion.
28. The combination cross-strap apparatus and brace according to embodiments 17-27, wherein: the hub portion provides a pair of spaced apart first loops positioned and configured for defining the first channel; and the hub portion further provides a pair of spaced apart second loops positioned and configured for defining the second channel.
29. The combination cross-strap apparatus and brace according to embodiments 17-28, wherein: the hub portion provides a pair of spaced apart first slots positioned and configured for defining the first channel; and the hub portion further provides a pair of spaced apart second slots positioned and configured for defining the second channel.
30. The combination cross-strap apparatus and brace according to embodiments 17-29, wherein: the at least one first engagement mechanism is positioned proximal at least one of the entry opening and exit opening of the first channel; and the at least one second engagement mechanism is positioned proximal at least one of the entry opening and exit opening of the second channel.
31. The combination cross-strap apparatus and brace according to embodiments 17-30, wherein: a first of the at least one first engagement mechanism is positioned proximal the entry opening of the first channel; a second of the at least one first engagement mechanism is positioned proximal the exit opening of the first channel; a first of the at least one second engagement mechanism is positioned proximal the entry opening of the second channel; and a second of the at least one second engagement mechanism is positioned proximal the exit opening of the second channel.

In closing, regarding the exemplary embodiments of the present invention as shown and described herein, it will be appreciated that a cross-strap apparatus for an osteoarthritis brace is disclosed and configured for joining a pair of cross-straps of the brace, opposite a hinge of the brace, and allowing for independent movement of the cross-straps while maintaining a load directly across a center of the user’s joint during movement of the joint. Because the principles of the invention may be practiced in a number of configurations beyond those shown and described, it is to be understood that the invention is not in any way limited by the exemplary embodiments, but is generally directed to a cross-strap apparatus for an osteoarthritis brace and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators – such as “first,” “second,” “third,” etc. – for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. §112(f) will begin with the words “means for,” but use of the term “for” in any other context is not intended to invoke treatment under 35 U.S.C. §112(f). Accordingly, Applicant reserves the right to pursue additional claims after filing this application, in either this application or in a continuing application.

It should be understood that the methods and the order in which the respective elements of each method are performed are purely exemplary. Depending on the implementation, they may be performed in any order or in parallel, unless indicated otherwise in the present disclosure.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicant and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A cross-strap apparatus for use with a brace, the brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint, the apparatus comprising:
a resilient hub portion sized and configured for engagement with each of the first and second cross-straps proximal to the cross-strap intersection; and
the hub portion providing an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection;
whereby, during use, the apparatus is capable of allowing for independent movement of the first and second cross-straps while maintaining a load directly across a center of the joint during movement of the joint.

2. The cross-strap apparatus of claim 1, wherein hub portion is constructed out of one or more of fabric, silicone, rubber, and soft plastic.

3. The cross-strap apparatus of claim 1, wherein:
the hub portion provides a pair of first engagement mechanisms positioned on opposing edges of the hub portion; and
the hub portion further provides a pair of second engagement mechanisms positioned on further opposing edges of the hub portion so as to be transversely oriented relative to the pair of first engagement mechanisms.

4. The cross-strap apparatus of claim 1, wherein:
the at least one first engagement mechanism is removably engageable with the first cross-strap; and
the at least one second engagement mechanism is removably engageable with the second cross-strap.

5. The cross-strap apparatus of claim 4, wherein each of the first and second engagement mechanisms provides hook material configured for engagement with corresponding loop material provided by each of the first and second cross-straps.

6. The cross-strap apparatus of claim 1, wherein:
the hub portion provides a first channel sized and configured for slidably receiving the first cross-strap therethrough; and
the hub portion further provides a second channel sized and configured for slidably receiving the second cross-strap therethrough so as to be transversely oriented relative to the first cross-strap.

7. The cross-strap apparatus of claim 6, wherein:
the first channel provides an entry opening sized and positioned for allowing a terminal end of the first cross-strap to pass therethrough as the first cross-strap is inserted into the first channel, and an opposing exit opening sized and positioned for allowing the terminal end of the first cross-strap to pass therethrough as the terminal end of the first cross-strap subsequently exits the first channel; and
the second channel provides an entry opening sized and positioned for allowing a terminal end of the second cross-strap to pass therethrough as the second cross-strap is inserted into the second channel, and an opposing exit opening sized and positioned for allowing the terminal end of the second cross-strap to pass therethrough as the terminal end of the second cross-strap subsequently exits the second channel.

8. The cross-strap apparatus of claim 7, wherein:
the hub portion comprises a first layer and an opposing second layer, the first and second layers cooperating to define the first and second channels therebetween;
whereby, during use of the apparatus, the cross-strap intersection is positioned between the first and second layers within the hub portion.

9. The cross-strap apparatus of claim 7, wherein:
the hub portion provides a pair of spaced apart first loops positioned and configured for defining the first channel; and
the hub portion further provides a pair of spaced apart second loops positioned and configured for defining the second channel.

10. The cross-strap apparatus of claim 7, wherein:
the hub portion provides a pair of spaced apart first slots positioned and configured for defining the first channel; and
the hub portion further provides a pair of spaced apart second slots positioned and configured for defining the second channel.

11. The cross-strap apparatus of claim 7, wherein:
the at least one first engagement mechanism is positioned proximal at least one of the entry opening and exit opening of the first channel; and
the at least one second engagement mechanism is positioned proximal at least one of the entry opening and exit opening of the second channel.

12. A cross-strap apparatus for use with a brace, the brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint, the apparatus comprising:
a resilient hub portion sized and configured for engagement with each of the first and second cross-straps proximal to the cross-strap intersection;
the hub portion providing an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection;
the hub portion providing a first channel sized and configured for slidably receiving the first cross-strap therethrough; and
the hub portion providing a second channel sized and configured for slidably receiving the second cross-strap therethrough so as to be transversely oriented relative to the first cross-strap in an overlapping fashion;
whereby, during use, the apparatus is capable of allowing for independent movement of the first and second cross-straps while maintaining a load directly across a center of the joint during movement of the joint.

13. A combination cross-strap apparatus and brace comprising:
a brace having an proximal portion engageable above a joint of a user, an opposing distal portion engageable below the joint, and a first cross-strap and second cross-strap extending between and interconnecting the proximal and distal portions, the first and second cross-straps transversely oriented relative to one another and positioned for intersecting one another in an overlapping fashion at a cross-strap intersection substantially at the joint; and
a cross-strap apparatus comprising:
a resilient hub portion sized and configured for engagement with each of the first and second cross-straps proximal to the cross-strap intersection; and
the hub portion providing an at least one first engagement mechanism positioned and configured for engagement with the first cross-strap, and an at least one second engagement mechanism positioned and configured for engagement with the second cross-strap, thereby maintaining the position of the hub portion relative to the cross-strap intersection;
whereby, during use, the apparatus is capable of allowing for independent movement of the first and second cross-straps while maintaining a load directly across a center of the joint during movement of the joint.

14. The combination cross-strap apparatus and brace of claim 13, wherein hub portion is constructed out of one or more of fabric, silicone, rubber, and soft plastic.

15. The combination cross-strap apparatus and brace of claim 13, wherein:
the hub portion provides a pair of first engagement mechanisms positioned on opposing edges of the hub portion; and
the hub portion further provides a pair of second engagement mechanisms positioned on further opposing edges of the hub portion so as to be transversely oriented relative to the pair of first engagement mechanisms.

16. The combination cross-strap apparatus and brace of claim 13, wherein:
the at least one first engagement mechanism is removably engageable with the first cross-strap; and
the at least one second engagement mechanism is removably engageable with the second cross-strap.

17. The combination cross-strap apparatus and brace of claim 13, wherein:
the at least one first engagement mechanism is permanently engaged with the first cross-strap; and
the at least one second engagement mechanism is permanently engaged with the second cross-strap.

18. The combination cross-strap apparatus and brace of claim 13, wherein:
the hub portion provides a first channel sized and configured for slidably receiving the first cross-strap therethrough; and
the hub portion further provides a second channel sized and configured for slidably receiving the second cross-strap therethrough so as to be transversely oriented relative to the first cross-strap.

19. The combination cross-strap apparatus and brace of claim 18, wherein:
the first channel provides an entry opening sized and positioned for allowing a terminal end of the first cross-strap to pass therethrough as the first cross-strap is inserted into the first channel, and an opposing exit opening sized and positioned for allowing the terminal end of the first cross-strap to pass therethrough as the terminal end of the first cross-strap subsequently exits the first channel; and
the second channel provides an entry opening sized and positioned for allowing a terminal end of the second cross-strap to pass therethrough as the second cross-strap is inserted into the second channel, and an opposing exit opening sized and positioned for allowing the terminal end of the second cross-strap to pass therethrough as the terminal end of the second cross-strap subsequently exits the second channel.

20. The combination cross-strap apparatus and brace of claim 19, wherein:
the at least one first engagement mechanism is positioned proximal at least one of the entry opening and exit opening of the first channel; and
the at least one second engagement mechanism is positioned proximal at least one of the entry opening and exit opening of the second channel.
